(19) United States
(12) Patent Application Publication
(10) Pub. No.: US 2014/0275773 A1
(43) Pub. Date: Sep. 18, 2014

(54) Delivery Catheter for Injecting a Substrate into a Tissue

(71) Applicants: The Regents of the University of California, Oakland, CA (US); CardioPolymers, Inc., Laguna Hills, CA (US)

(72) Inventors: Norman Tarazona, Duluth, GA (US); Randall J. Lee, Hillsborough, CA (US); Manoj B. Raghuraman, Irvine, CA (US); Gary H. Sanders, Rancho Santa Margarita, CA (US)

(73) Assignees: THE REGENTS OF THE UNIVERSITY OF CALIFORNIA, Oakland, CA (US); CARDIOPOLYMERS, INC., Laguna Hills, CA (US)

(21) Appl. No.: 14/211,615

(22) Filed: Mar. 14, 2014

Related U.S. Application Data
(60) Provisional application No. 61/801,625, filed on Mar. 15, 2013.

(51) Int. Cl. A61M 5/42; A61B 1/04

(52) U.S. Cl. CPC A61M 5/425; A61B 1/04
USPC 600/106; 604/506; 604/115; 604/131

(57) ABSTRACT

A catheter device is useful in a procedure in which an injectable material or device is injected into a tissue of a patent. In one implementation, for example, the catheter device is useful in injecting a compound into a tissue of the heart, such as the myocardium. The distal tip portion of the catheter may include an extensible and retractable needle in combination with a camera, a balloon, a vacuum port, or any combination thereof.

### CROSS-REFERENCE TO RELATED APPLICATIONS

This application claims the benefit of U.S. Provisional Patent Application Ser. No. 61/801,625 filed Mar. 15, 2013, which hereby is incorporated herein in its entirety by reference thereto.

### BACKGROUND OF THE INVENTION

1. Field of the Invention

The instant invention relates a catheter device for use in a procedure in which an injectable material or device is injected into a tissue of a patent. In one implementation, for example, the catheter device is useful in injecting a compound into a tissue of the heart, such as the myocardium of the heart.

2. Description of Related Art

Injection of various materials into the myocardium of the heart while the heart is beating is desirable. Various materials and techniques are disclosed in, for example, U.S. Patent Application Publication No. US 2008/0065046 published Mar. 13, 2008 in the name of Hani N. Sabbah et al. and entitled “Intramyocardial Patterning for Global Cardiac Resizing and Reshaping.”

### BRIEF SUMMARY OF THE INVENTION

One embodiment of the present invention is a method of injecting a needle into a tissue surface in a patient comprising: providing a delivery catheter comprising a distal tip portion and a proximal handle portion, the distal tip portion comprising an extensible and retractable needle and a suction port in fluid communication with a vacuum port of the proximal handle portion, the vacuum port adapted to create suction at the suction port when a vacuum is applied to the vacuum port; directing the distal tip portion of the delivery catheter to a treatment site; applying a vacuum to the vacuum port to create suction at the suction port of the distal tip portion and engage a tissue surface at a first location displaced from a second location of the tissue surface to be treated; and extending the needle from the distal tip portion into the tissue surface at the second location of the tissue surface.

Another embodiment of the present invention is an injection catheter comprising: a proximal handle portion comprising a vacuum port; a delivery catheter coupled to the proximal handle portion and comprising a distal tip portion, the distal tip portion comprising: an extensible and retractable needle, a tip deflector configured to deflect the needle at an angle displaced from a longitudinal axis of the distal tip portion, and a suction port in fluid communication with a vacuum port of the proximal handle portion, the vacuum port adapted to create suction at the suction port when a vacuum is applied to the vacuum port, wherein the suction port is configured to engage a tissue surface to be injected at a first location and the needle is configured to extend from the distal tip portion at the angle and extend into the tissue surface at a second location displaced from the first location.

Another embodiment of the present invention is an injection catheter comprising: a proximal handle portion comprising a vacuum port and a needle hub; a delivery catheter coupled to the proximal handle portion and comprising a distal tip portion, the distal tip portion comprising: an extensible and retractable needle in fluid communication with the needle hub, a tip deflector configured to deflect the needle at an angle displaced from a longitudinal axis of the distal tip portion, and a suction port in fluid communication with a vacuum port of the proximal handle portion, the vacuum port adapted to create suction at the suction port when a vacuum is applied to the vacuum port, wherein the suction port is configured to engage a tissue surface to be injected to stabilize the distal tip portion adjacent the tissue surface and the needle is configured to extend from the distal tip portion at the angle, extend into the tissue surface at a location on of the tissue surface outside of an engagement region between the suction port and the tissue surface.

Another embodiment of the present invention is an injection catheter comprising: a proximal handle portion comprising a vacuum port; a delivery catheter coupled to the proximal handle portion and comprising a distal tip portion, the distal tip portion comprising: an extensible and retractable needle, wherein the needle is configured to deflect at an angle displaced from a longitudinal axis of the distal tip portion, and a suction port in fluid communication with a vacuum port of the proximal handle portion, the vacuum port adapted to create suction at the suction port when a vacuum is applied to the vacuum port, wherein the suction port is configured to engage a tissue surface to be injected at a first location and the needle is configured to extend from the distal tip portion at the angle and extend into the tissue surface at a second location displaced from the first location.

Another embodiment of the present invention is an injection catheter comprising: a proximal handle portion comprising fluid port; a delivery catheter coupled to the proximal handle portion and comprising a distal tip portion, the distal tip portion comprising: an extensible and retractable needle, a tip deflector configured to deflect the needle in a first direction at an angle displaced from a longitudinal axis of the distal tip portion, and a balloon coupled to the distal tip portion and in fluid communication with the fluid port, the balloon configured to be deployed in response to fluid delivered to the fluid port of the proximal handle portion and delivered to the balloon to extend the balloon in a second direction distinct from the first direction and to bias or displace the needle toward a tissue surface to be treated.

The foregoing and other aspects, features, details, utilities, and advantages of the present invention will be apparent from reading the following description and claims, and from reviewing the accompanying drawings.

### BRIEF DESCRIPTION OF THE SEVERAL VIEWS OF THE DRAWINGS

FIG. 1 shows a top view of a catheter device.

FIG. 2 shows a side view of the catheter device of FIG. 1.

FIG. 3 shows a front end view of the catheter device of FIGS. 1 and 2 taken facing a distal end of a delivery catheter of the catheter device.

FIG. 4 shows a rear view of the catheter device of FIGS. 1-3 taken facing a proximal handle end of the catheter device.

FIG. 5 shows various section lines superimposed on the view of FIG. 3.

FIG. 6 shows a first section view of the catheter of FIGS. 1-5 taken along section line A-A.

FIG. 7 shows a second section view of the catheter of FIGS. 1-5 taken along a second section line B-B.

FIG. 8 shows a section view of a tip of the catheter of FIGS. 1-7 showing an injection needle extending from the tip of the catheter.

FIG. 9 shows a section view of the tip of the catheter shown in FIG. 8 without the injection needle in lumens of the catheter tip.

FIG. 10 shows a side view of a tip deflector for use in deflecting a needle in an angular direction from the catheter tip.

FIG. 11 shows a bottom view of the suction port disposed in the catheter tip.

FIG. 12 shows a balloon stabilization component deployed from the catheter tip.

FIG. 13 shows an example implementation of a catheter device being used to inject a substance into the myocardium of a heart when the tip of the catheter device is disposed between the myocardium and the pericardial sac.

FIG. 14 shows a section view of a balloon stabilizing component in a deployed configuration.

FIG. 15 shows an alternative tip flap stabilization component to a balloon stabilization component.

### DETAILED DESCRIPTION OF THE INVENTION

FIGS. 1 and 2 show a top view and a side view of an example implementation of a catheter device 10. FIG. 3 shows a front end view of the catheter device of FIGS. 1 and 2 taken facing a distal end of a delivery catheter of the catheter device. FIG. 4 shows a rear view of the catheter device of FIGS. 1-3 taken facing a proximal handle end of the catheter device. FIG. 6 shows a first section view of the catheter of FIGS. 1-5 taken along section line A-A. FIG. 7 shows a second section view of the catheter of FIGS. 1-5 taken along a second section line B-B. Section lines A-A and B-B are shown in FIG. 5.

The catheter device 10 includes a handle portion 12 and a delivery catheter 14. The handle portion 12 of the catheter device 10 includes a plurality of connection points, such as ports, hubs, and connectors for coupling one or more devices to the handle portion 12 of the catheter device 10.

In the particular implementation shown in FIGS. 1 and 2, for example, the handle portion 12 of the catheter device 10 includes connectors for a vacuum port 16 for providing suction to the delivery catheter 14, a needle hub 18 for coupling an injection needle system to an injection needle 26 disposed at the distal tip 24 of the delivery catheter 14, and a camera lead 20 for coupling a fiber optic or other link to a camera 22 disposed generally at the distal tip 24 end of the delivery catheter 10.

The connectors are operably coupled to one or more devices disposed at the distal tip 24 of the delivery catheter 14. The vacuum port 16, for example, is operably coupled to a suction port 28 disposed along a side edge of the distal tip 24 of the delivery catheter 14. In one implementation, the vacuum port 16 and the suction port 28 are coupled via one or more lumens extending from the vacuum port 16 in the handle portion 12 through the delivery catheter 14 to the distal tip 24 of the delivery catheter 14. As a vacuum is drawn at the vacuum port 16 of the catheter device 10, the suction port 28 at the distal end 24 of the delivery catheter 14 is in fluid communication with the vacuum port 16, and suction is imparted to the suction port 28. As described in more detail below, the suction may be used to stabilize the distal tip 24 of the delivery catheter 14, to evacuate fluid or debris from the area in which the tip of the catheter is disposed, and/or to deliver a fluid (e.g., a saline wash or saline with contrast) to an area near the tip 24 of the delivery catheter 14.

Similarly, a retractable and extensible injection needle 26 disposed at the distal tip 24 of the delivery catheter 14 is operatively coupled to the needle hub 18. In one implementation, for example, the injection needle 26 is in fluid communication with the needle hub for delivering one or more injectable polymer, cells, drugs, device, biologics or any combination thereof to a location adjacent the distal tip 24 of the delivery catheter 14. The injection needle 26 may be in fluid communication with the needle hub 18 via a lumen, via a needle cannula, or other fluid communication path extending from the needle hub 18 of the handle portion 12 through the delivery catheter 14 to the needle 26 extending from the tip 24 of the delivery catheter 14. The lumen, cannula, hyper needle or other fluid communication path can be designed to reduce or minimize a pressure level required to deliver an injectable material to the needle. A diameter or width of the fluid communication path sufficient to allow the injectable material to flow through the path from the needle hub 18 to the injection needle 26 depending on the characteristics of the injectable material. The dimensions of the fluid communication path may be different depending on the viscosity or other characteristics of the injectable material to ensure that the material is able to flow to the injection needle 26 without overwhelming resistance. The fluid communication path may be larger for a relatively viscous material, such as an injectable polymer, than for an injectable saline solution, for example. In one implementation, for example, an internal diameter of a needle lumen (see, e.g., needle lumen 48 in FIG. 9) or cannula is about 1.3 mm to about 1.4 mm at a proximal end at the handle portion and about 0.3 mm to about 0.6 mm at the distal tip.

During delivery of the delivery catheter 14, the injection needle 26 can be retracted within the tip 24 of the delivery catheter 14 to prevent the needle from reducing the maneuverability of the catheter and to prevent harm to a patient. Once the tip 24 of the delivery catheter is at a desired site, the injection needle 26 can be extended from the tip 24 of the catheter and injected into a tissue of a patient. As shown in FIGS. 2 and 3, the needle 26 extends from the tip 24 of the delivery catheter 14 at a downward angle toward an injection surface located under the suction port 28 disposed on the tip 24 of the delivery catheter 14. Once the needle 26 is extended into tissue, an injection can be performed in which an injectable is delivered into the tissue under pressure from the needle hub 18.

Although not drawn to exact scale, FIG. 2 shows the injection needle 26 partially extended from the tip 24 of the delivery catheter 14. In this example, the needle is extending at an angle towards an injection surface and generally toward the suction port disposed on a bottom surface of the catheter tip 24. The angle the injection needle 26 extends from the catheter tip 24 can be customized depending on the procedure to be performed. Where the injection needle 26 is to be inserted into a myocardium wall of the heart, for example, the needle can extend downwards from the tip 24 of the delivery catheter 14 at an angle Θ between about 30 degrees and about 60 degrees from the longitudinal axis of the distal tip of the delivery catheter. In one particular implementation, for example, the injection needle 26 extends down from the tip 14 at an angle Θ of about 45 degrees.

The angulation of the injection needle 26 may be accomplished in a number of ways. In one example implementation, for example, a stainless steel or other rigid needle may be deflected by a tip deflector 32, or other rigid surface, such as shown in FIGS. 8 and 10. In this implementation, as the needle 26 is extended from the tip 24 of the catheter 14, the tip deflector 32 or other rigid surface deflects the needle 26 at a predetermined angle downward from the tip 24 toward an underlying tissue. The tip deflector 32 or other surface is rigid enough to prevent the needle from penetrating the deflector and forces the needle in the desired direction as it is extended from the tip 24.

In another example implementation, a shape memory alloy needle, such as a Nitinol needle, may be pre-bent at an angle and then straightened when placed in the delivery catheter 14. A structure of the catheter, such as a lumen, maintains the shape memory alloy material in a straight configuration. However, when the needle is extended outside of the lumen or other structure (e.g., a hyper needle) of the catheter, the shape memory alloy needle reverts to its pre-bent state and can be angled downwards past the suction port disposed on a lower surface of the tip of the catheter. In some implementations, the tip deflector or another structure in the tip of the catheter can direct the shape memory alloy in the correct direction. In other implementations, however, the shape memory alloy needle can be oriented within the catheter so that, upon its resumption of the pre-bent shape, it is already oriented in the predetermined angle and direction.

In implementations where the suction port 28 stabilizes the tip 24 of the catheter 14 by engaging a surface such as a tissue of a patient, the injection needle 26 can be extended beyond an outer dimension of the suction port 28 disposed on a bottom edge of the catheter tip 26 to inject the needle into the adjacent tissue of a patient displaced (e.g., laterally displaced) from the location on the tissue surface where the suction port is engaging the tissue. Thus, the needle is able to be inserted into the tissue at a location outside of where the suction port is engaging the tissue. In some procedures, for example, the injection needle 26 may be extensible into the tissue from about 3 mm to about 5 mm to inject a material or other injectable into the tissue. Depending on the particular procedure, however, the injection needle may be designed to extend any desired distance into the tissue.

In one implementation, the needle may include a locking mechanism, such as a luer lock system disposed at a proximal end (near the handle portion 12) that would prevent or reduce backwards movement of the needle during operation, such as due to movements within a beating heart.

The camera 22 is also mounted to the tip 24 of the delivery catheter 14 so that the operation of the needle as well as movement of the catheter is captured by the camera and communicated back through the camera lead 20 for display to a surgeon operating the catheter device 10 providing visible feedback for the surgeon. In one implementation, for example, the camera 22 comprises a CMOS camera with a fiber optic link communicating through a lumen of the delivery catheter 14 to the camera lead 20 extending from the handle portion of the catheter device for display on a monitor where it may be viewed during operation of the catheter device 10. In one implementation, an illumination device may also be used in cooperation with the camera 22. The illumination device may be incorporated with the camera or may be separate from the camera and disposed at the tip of the delivery catheter to illuminate a region near the tip during a procedure.

The handle portion 12 further includes a steering device 30. In the particular implementation shown in FIGS. 1-7, for example, the steering device 30 comprises a pair of opposing steering levers 32 operable by a surgeon to steer the delivery cathode 14 during a procedure. As described above, the surgeon may use the steering device 30 in conjunction with the camera 22 or may monitor the progress of the delivery catheter utilizing one or more radiopaque markers in combination with the catheter. Although a particular steering device 30, 32 is shown in this example, any catheter steering mechanism may be used in other implementations.

Section views shown in FIGS. 6 and 7 show various lumens, leads and cannulas coupling the connectors of the handle 12 of the catheter device with components disposed at the tip of the delivery catheter. FIG. 6, taken along section line A-A, shows the camera lead 20 extending from the handle portion 12 of the catheter device 10 to a camera 22 disposed at the tip 24 of the delivery catheter 14. FIG. 6 further shows a needle cannula 34 coupling the needle hub 18 with the injection needle 26 that is extensible from and retractable into the tip of the delivery catheter.

FIG. 7 further shows guide wires 36 and 38 used in conjunction with the steering levers 32 to guide the delivery catheter during a procedure.

FIG. 8 shows a section view of a tip of the catheter of FIGS. 1-7. FIG. 8 shows an injection needle 26 extending from the tip of the catheter. FIG. 9 shows a section view of the tip of the catheter shown in FIG. 8 without the injection needle in lumens of the catheter tip.

As shown in FIG. 8, an injection needle 26, such as a stainless steel needle or a shape memory alloy (e.g., Nitinol), is coupled to a needle cannula 27 that extends back to a needle hub 18 (see, e.g., FIGS. 1 and 2) and provides fluid communication for an injectable material to be delivered to the needle 26 for injection into a patient during a procedure. The needle cannula 27 extends through a lumen of the delivery catheter 14 and handle portion 12 of the catheter device 10.

An example implementation of a tip deflector is shown in detail in FIGS. 8 and 10. In this implementation, the tip deflector 32 comprises a hollow, at least partially rigid tube that extends into a lumen housing the needle and needle cannula. The tip deflector also extends along the upper tip of the delivery catheter and angles downwardly at the tip, directing the needle when it is extended toward a target tissue.

FIGS. 8, 9 and 11 further show an example implementation of a suction port 28. In this implementation, the suction port 28 comprises a suction lip or flange 40 defining an opening 42 in the tip 24 of the delivery catheter 14. FIG. 11 shows one example of a generally oval suction lip 40 defining the opening 42 of the suction port 28, although other shapes and configurations are possible. The opening 42 of the suction port 28 is in fluid communication with a vacuum lumen 44 that is coupled to the vacuum port 16. Thus, when a vacuum is applied to the vacuum port 16, the evacuated pressure in the vacuum lumen 44 creates a suction effect at the opening 42 of the suction port 28.

In use, the suction created at the suction port 28 stabilizes the tip 24 of the delivery catheter 14 by holding the suction lip 40 in contact with a tissue surface of a patient. In one implementation, for example, the tissue surface comprises a myocardium of a heart and the suction port can stabilize the tip 24 between the myocardium and the pericardial sac of the heart.

FIGS. 8 and 9 further show a camera lumen 46 through which the camera lead 20 extends between the tip 24 of the delivery catheter 14 and the handle portion 12 of the catheter device 10. The particular arrangement of the lumens, cannulas and leads extending through the delivery catheter 14 and the handle portion are merely examples of possible configurations. Other configurations are also possible.

FIG. 12 shows an example implementation of a balloon stabilizing component 50 that may be coupled to the tip 24 of the delivery catheter 14. In this implementation, the balloon 50 is delivered to a treatable location in a deflated configuration against the tip 24 of the delivery catheter. When the tip of the delivery catheter has been moved to a treatment site, the balloon can be deployed by inflating the balloon via a lumen or other channel or device in fluid communication with a port or connected located in the handle portion 12 of the catheter device 10. In the particular implementation shown in FIG. 11, for example, the balloon is deployed away from a top region of the tip 24. The deployment of the balloon may come into contact with a tissue surface of a patient and move or bias the tip 24 away from that tissue surface and further move the needle closer to and/or stabilize the needle with respect to a treatment/injection surface located in a direction generally opposite of the direction the balloon is deployed.

In various implementations, the catheter device 10 may include a suction port 28 stabilization device and/or a balloon stabilization device to stabilize the tip 24 of the delivery catheter during a procedure. Where both a suction port stabilization device and a balloon stabilization component are provided, an operator may decide whether to use one or both of the stabilizing components depending on the circumstances of the procedure.

FIG. 13 shows an example implementation of a catheter device being used to inject a substance into the myocardium of a heart when the tip of the catheter device is disposed between the myocardium and the pericardial sac. In this implementation, the catheter device includes both a balloon 66 and a vacuum cup/suction port 64 stabilization components arranged on opposing sides of the tip of the delivery catheter. In this implementation, a procedure in which an injectable material (e.g., Algisyl-LVR® material) is injected into the myocardium of the heart is being performed. The tip 24 of the delivery catheter is extended to a position between the pericardial sac 61 and the myocardium 60 of the heart. Depending on the anatomical features of the location of an injection, an operator may decide that one or both of the stabilizing components would better stabilize the tip 24 of the delivery catheter before and during the injection. If the pericardial sac is tightly pressing the tip of the catheter against the myocardium, for example, the operator may decide not to deploy the balloon and rely on the suction port to keep the tip in place during an injection.

In the example shown in FIG. 13, however, the balloon stabilization component 66 is deployed in a direction opposite the suction port 64. As the balloon 66 inflates, it pushes against the pericardial sac 61 and in reaction biases or moves the tip 24 of the delivery catheter 62 toward the myocardium 60. As the suction port 64 moves closer to the myocardium 60, the suction port 64 engages with a surface of the myocardium 60 and stabilizes the tip against the myocardium 60 surface. In this implementation, the balloon 66 assists the suction port 64 in engaging a tissue surface 60 and in maintaining an engagement with the surface. Once the tip of the catheter 62 is successfully stabilized, the operator extends the injection needle 68 into the surface of the myocardium 60.

FIG. 14 shows a section view of a balloon stabilizing component 50 in an expanded configuration. An interior of the balloon 50 is in fluid communication with a balloon lumen 52 that extends through the delivery catheter 14 to the handle portion 12. The balloon 50 may be inflated via a fluid, e.g., air or a liquid such as saline, pushed through the balloon lumen 52.

FIG. 15 shows an alternative tip flap 60 stabilization component to a balloon stabilization component. In this implementation, a tip flap 60 can be deployed via a lever mechanism located beneath the flap 60. The flap 60, for example, may be deployed by use of a lever or other similar mechanical device.

Example Surgical Procedure

In one particular implementation, a minimally invasive procedure to deliver a compound, such as the Algisyl-LVR® material, to the myocardium through the epicardial space in a beating heart procedure is performed using the catheter device 10. Although this example surgical procedure discloses injecting a particular compound, the Algisyl-LVR® material, the use of the catheter device 10 is not so limited. As discussed above, the catheter device 10 may be used to inject any injectable material or device, such as but not limited to any substrate such as cells, drugs, biologics, devices or any combination thereof. Example surgical operations include any combination or sub-combination of the following.
An initial angiogram (or other diagnostic) is performed to provide a baseline of the left coronary artery system in a patient.
A small incision is made just below the sternum and above the diaphragm to gain sub-xiphoid entry.
Once entry is made into the anterior space, a Touhy needle with stylet is used to puncture the pericardial sac and entry into the pericardial is achieved. In one implementation, this can be verified by introducing an 0.035 j-tipped guide wire.
A contrast dye is injected into this space to achieve fluoroscopic visualization.
Dilators in increasing size are then used to widen the incision to allow for a guide catheter.
A steerable or a non-steerable guide catheter that is either straight or pre-shaped may be used to provide directionality to the delivery catheter. Alternatively, the delivery catheter may be introduced without the assistance of a guide catheter over a guide wire.
After the guide catheter is in place, the minimally invasive delivery catheter is introduced. The delivery catheter will contain a camera, a needle, and at least one of a suction port/vacuum cup and one or more stabilization balloon component(s).
One or more injection sites on the left ventricle (LV) are identified on a fluoroscopy screen using the baseline angiogram or other diagnostic.
The delivery catheter with or without the help of the guide catheter then navigates to the most posterior injection site on the LV.
Excess fluid, blood or debris may be removed by engaging vacuum to create a suction at a suction port on the tip of the delivery catheter.
Once an injection site is confirmed using camera visualization and/or fluoroscopy, then a needle is primed (e.g., ex-vivo) with Algisyl-LVR® material (or another injectable material or device such as a substrate such as cells, drugs, biologics, devices or any combination there-of) and the needle is then inserted through a delivery catheter needle lumen until the needle reaches the tip located at the distal end of the delivery catheter.
One or more balloon is deployed and then a vacuum is applied to a vacuum port of the catheter device to create suction at a suction port disposed on the tip of the delivery catheter. In one implementation, for example, the balloon is deployed against the pericardial sac and biases the tip of the delivery catheter (and the suction port disposed on the tip) toward the myocardium of the heart. This moves the suction port of the delivery catheter adjacent the myocardium and assists the suction port to engage the myocardium via suction. In other implementations, however, only the balloon may be deployed or only the suction port/vacuum cup may be engaged as determined by the operator.
Once the delivery catheter is stabilized either via balloon, vacuum or both, the needle is then advanced through a tip deflector and penetrates the myocardium. This is confirmed via camera visualization. An injectable material, such as Algisyl-LVR (or substrate), is then injected through the needle and into the myocardium. The operator may continue to monitor the process of the injection using the camera to determine whether the needle remains in the injection site of the myocardium for the duration of the injection.
Once the required injection volume is delivered, then needle is retracted. Lack of leakage of injectate is confirmed via camera visualization.
The vacuum is disengaged at the vacuum port releasing the suction port from the myocardium. If a balloon has been deployed, the balloon is retracted. The delivery catheter is then steered to the next injection site
The operations of identifying an injection site, stabilizing the tip of the delivery catheter, extending the needle, injecting a substrate (e.g., the Algisyl-LVR® material), retracting the needle and releasing the tip of the delivery catheter can be repeated without having to prime the needle with the Algisyl-LVR® material (or another substrate). New needle priming is only performed if determined to be beneficial by the operator.
Once the Algisyl-LVR® material (or other substrate) is delivered to all injection sites, then the delivery catheter is retracted.
A guide catheter is retracted if applicable.
Closure of incision is performed and procedure is complete.

Although embodiments of this invention have been described above with a certain degree of particularity, those skilled in the art could make numerous alterations to the disclosed embodiments without departing from the spirit or scope of this invention. All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

1. A method of injecting a needle into a tissue surface in a patient comprising:
providing a delivery catheter comprising a distal tip portion and a proximal handle portion, the distal tip portion comprising an extensible and retractable needle and a suction port in fluid communication with a vacuum port of the proximal handle portion, the vacuum port adapted to create suction at the suction port when a vacuum is applied to the vacuum port;
directing the distal tip portion of the delivery catheter to a treatment site;
applying a vacuum to the vacuum port to create suction at the suction port of the distal tip portion and engage a tissue surface at a first location displaced from a second location of the tissue surface to be treated;
extending the needle from the distal tip portion into the tissue surface at the second location of the tissue surface.

2. The method of claim 1 further comprising injecting a material through the needle into the tissue surface.

3. The method of claim 2 further comprising retracting the needle away from the tissue surface into the distal tip portion of the delivery catheter.

4. The method of claim 1 wherein the needle is injected at an angle deflected from a longitudinal axis of the distal tip portion.

5. The method of claim 4 wherein the angle in between about 20 degrees and about 70 degrees.

6. The method of claim 1 wherein the suction port comprises a lip surrounding and defining an opening in the distal tip portion of the delivery catheter.

7. The method of claim 1 wherein a balloon is deployed in a generally opposing direction from the suction port.

8. The method of claim 7 wherein the tissue surface is a myocardium of a heart.

9. The method of claim 8 wherein the balloon is deployed against a pericardial sac.

10. An injection catheter comprising:
a proximal handle portion comprising a vacuum port;
a delivery catheter coupled to the proximal handle portion and comprising a distal tip portion, the distal tip portion comprising:
an extensible and retractable needle,
a tip deflector configured to deflect the needle at an angle displaced from a longitudinal axis of the distal tip portion, and
a suction port in fluid communication with a vacuum port of the proximal handle portion, the vacuum port adapted to create suction at the suction port when a vacuum is applied to the vacuum port,
wherein the suction port is configured to engage a tissue surface to be injected at a first location and the needle is configured to extend from the distal tip portion at the angle and extend into the tissue surface at a second location displaced from the first location.

11. The injection catheter of claim 10 wherein the extensible and retractable needle is in fluid communication with a needle hub for receiving an injectable material.

12. The injection catheter of claim 10 wherein the tip deflector is configured to deflect the needle between about 30 and 60 degrees from the longitudinal axis of the distal tip portion.

13. The injection catheter of claim 10 further comprising a balloon coupled to the distal tip portion and in fluid communication with a fluid port disposed at the proximal handle portion, the balloon configured to be deployed in response to fluid delivered to the fluid port of the proximal handle portion and delivered to the balloon to extend the balloon in a generally opposing direction from the suction port and to bias or displace the needle toward a tissue surface to be treated.

14. The injection catheter of claim 13 wherein the fluid comprises a liquid or a gas.

15. The injection catheter of claim 10 wherein the needle comprises a shape memory alloy.

16. The injection catheter of claim 15 wherein the needle is pre-bent.

17. An injection catheter comprising:
a proximal handle portion comprising a vacuum port and a needle hub;
a delivery catheter coupled to the proximal handle portion and comprising a distal tip portion, the distal tip portion comprising:
an extensible and retractable needle in fluid communication with the needle hub,
a tip deflector configured to deflect the needle at an angle displaced from a longitudinal axis of the distal tip portion, and
a suction port in fluid communication with a vacuum port of the proximal handle portion, the vacuum port adapted to create suction at the suction port when a vacuum is applied to the vacuum port,
wherein the suction port is configured to engage a tissue surface to be injected to stabilize the distal tip portion adjacent the tissue surface and the needle is configured to extend from the distal tip portion at the angle, extend into the tissue surface at a location on of the tissue surface outside of an engagement region between the suction port and the tissue surface.

18. An injection catheter comprising:
a proximal handle portion comprising a vacuum port;
a delivery catheter coupled to the proximal handle portion and comprising a distal tip portion, the distal tip portion comprising:
an extensible and retractable needle, wherein the needle is configured to deflect at an angle displaced from a longitudinal axis of the distal tip portion, and
a suction port in fluid communication with a vacuum port of the proximal handle portion, the vacuum port adapted to create suction at the suction port when a vacuum is applied to the vacuum port,
wherein the suction port is configured to engage a tissue surface to be injected at a first location and the needle is configured to extend from the distal tip portion at the angle and extend into the tissue surface at a second location displaced from the first location.

19. An injection catheter comprising:
a proximal handle portion comprising fluid port;
a delivery catheter coupled to the proximal handle portion and comprising a distal tip portion, the distal tip portion comprising:
an extensible and retractable needle,
a tip deflector configured to deflect the needle in a first direction at an angle displaced from a longitudinal axis of the distal tip portion, and
a balloon coupled to the distal tip portion and in fluid communication with the fluid port, the balloon configured to be deployed in response to fluid delivered to the fluid port of the proximal handle portion and delivered to the balloon to extend the balloon in a second direction distinct from the first direction and to bias or displace the needle toward a tissue surface to be treated.

20. The device of claim 19 further comprising a suction port in fluid communication with a vacuum port of the proximal handle portion, the vacuum port adapted to create suction at the suction port when a vacuum is applied to the vacuum port.

21. The device of claim 19 wherein the suction port is configured to engage a tissue surface to be injected.

22. The device of claim 19 further comprising a camera disposed on the distal tip portion and coupled to the proximal handle portion for providing an image of an area adjacent the distal tip portion.
